(19) United States
(12) Patent Application Publication
(10) Pub. No.: US 2018/0303559 A1
(43) Pub. Date: Oct. 25, 2018

(54) ELECTRONIC POSITION GUIDANCE DEVICE WITH REAL-TIME AUDITORY AND VISUAL FEEDBACK

(71) Applicant: NEW YORK UNIVERSITY, New York, NY (US)

(72) Inventors: Timothy SHEPHERD, Tarrytown, NY (US); Nainesh PARIKH, New York, NY (US); Peter THELWALL, Newcastle upon Tyne (GB)

(21) Appl. No.: 15/769,315

(22) PCT Filed: Oct. 18, 2016
(86) PCT No.: PCT/US16/57558

§ 371 Date: Apr. 18, 2018

Related U.S. Application Data
(60) Provisional application No. 62/243,478, filed on Oct. 19, 2015.

(51) Int. Cl. A61B 34/20; A61B 34/00

(52) U.S. Cl. CPC A61B 34/20; A61B 2034/2051; A61B 2034/2048; A61B 34/25

(57) ABSTRACT

An electronic position guidance device is configured to attach to a medical device having a first end configured for percutaneous insertion and a second end configured to remain exterior to a patient's skin. The device includes an orientation attachment configured to detect an orientation and a position of the medical device, a processor configured to receive orientation and position information from the orientation attachment and determine a degree to which an actual insertion angle of the medical device deviates from a target angle and/or a degree to which an actual insertion depth of the medical device deviates from a target insertion depth, a display configured to visually convey to a user, in real-time via graphic, text and/or color, the orientation and the position of the medical device, and a speaker configured to audibly convey to the user, in real-time via sound, the orientation and the position of the medical device.

### CROSS-REFERENCE TO RELATED APPLICATIONS

The present application is the U.S. national phase application under 35 U.S.C. § 371 of International Application No. PCT/US2016/057558 filed Oct. 18, 2016, which claims the benefit of and priority to U.S. Provisional Patent Application No. 62/243,478, filed Oct. 19, 2015, the entire disclosures of which are incorporated herein by reference.

### FIELD OF THE INVENTION

The present invention relates generally to the field of position guidance devices for minimally invasive medical procedures. More specifically, the present invention relates to an electronic position guidance device and method that allows the user to actively adjust an orientation (e.g., direction/angle) of the needle relative to the vertical, with the assistance of dynamic, real time auditory and visual feedback mechanisms. Conversely, the feedback from the invention can be used to maintain a stable position for a medical device in 3-dimensional space without physical stereotaxis.

### BACKGROUND

This section is intended to provide a background or context to the invention recited in the claims. The description herein may include concepts that could be pursued, but are not necessarily ones that have been previously conceived or pursued. Therefore, unless otherwise indicated herein, what is described in this section is not prior art to the description and claims in this application and is not admitted to be prior art by inclusion in this section.

Minimally invasive medical procedures with image guidance for needle access to anatomic structures are becoming increasingly common and important to the clinical management of patients, particularly because they represent minimally invasive alternatives to surgical interventions. As illustrated in FIGS. 1A-1F, image guidance is typically used for the vast majority of percutaneous procedures, for example: CT-guided pulmonary nodule biopsy and/or ablation (A), retroperitoneal lymph node biopsy (B) or bone biopsy (C), intra-abdominal or intra-pelvic abscess drainage (D) or ablation procedures of tumors of visceral abdominal organs (liver or kidney tumors) (E). For percutaneous procedures that utilize other imaging modalities, such as fluoroscopy, precise image guidance is paramount; for example during lumbar puncture (F, AP projection) for cerebrospinal fluid studies, intrathecal chemotherapy or CT myelogram. In 2008, the annual rate of image-guided biopsies increased to 1.945 per 100,000 Medicare enrollees (almost 2% per capita). See Kwan et al., “Effect of Advanced Imaging Technology on How Biopsies Are Done and Who Does Them,” 256(3) Radiology 751 (2010), the entire contents of which are hereby incorporated by reference. For example, the New York University Department of Radiology & Biomedical Imaging currently performs 50-100 image-guided procedures each week. These image-guided procedures generate significant revenue for hospitals and physician practices.

CT image guidance improves visualization of the tissue target during a variety of medical interventions including biopsies, radiofrequency ablations, pain procedures and other interventions. CT-guided procedures are minimally invasive, can reach small deep tissue structures in or surrounded by bone, require minimal patient recovery, and decrease healthcare costs and immediately impact clinical management. However, CT-guided procedures still risk inadvertent tissue injury and have longer procedure times than fluoroscopy or ultrasound-guided procedures. Moreover, a patient and his/her clinical care team may be concerned regarding the associated radiation use in CT-guided procedures. These potential limitations are mitigated by operator training, skill and experience performing CT-guided procedures. The most common current practice for CT-guided procedures involves iterative readjustment of needle position with focused repeat CT imaging of the patient. The above discussion also applies in the growing field of MRI-guided interventions, including but not limited to high-frequency ablation procedures (“HIFU”).

Percutaneous image-guided procedures share a common protocol regardless of imaging modality utilized for guidance: after initial images are obtained, an operator determines a safe surface entry point, trajectory angle and penetration distance for a manually-directed needle to reach a target organ or tissue. As an example, in CT-guided procedures, establishing the best surface entry point for the procedure works well using a standard metallic fiducial and grid (see FIG. 2) and penetration distance is easy to measure on scout images. For example, FIG. 2 illustrates a typical process for a CT-guided procedure demonstrated for a left L4 transforaminal epidural block for lower back pain. In this procedure, a patient is placed prone, and then CT images are obtained in a region of interest with a radio-opaque grid on a surface of the patient (“1^{st} Scout”). Operators use these images to plan an optimal needle trajectory to reach an anatomic target which includes consideration of needle angle and depth. The surface entry for the needle is verified with a metallic fiducial bead (“2^{nd} Scout”). The operator then places the needle, which is slowly advanced (“Guide”) and adjusted as needed using CT guidance until the target is reached and may be verified with contrast injection (“Contrast”). Although the protocol for establishing a target trajectory angle and penetration distance is common, prescribing and maintaining a correct needle angle is more challenging in daily practice. This is due in part to the fact that image orientations generated by the CT scanner, for example, are relative to the scanner and procedure room floor. Thus, the operator plans the angles with respect to the images, as opposed to planning the angles with respect to the patient who might be positioned in a slight oblique orientation to the scanner or floor to enhance their comfort.

FIG. 3 illustrates an example of an appropriate angle for needle position prior to penetrating the surface of the patient in terms of target angles and horizon angles. In particular, FIG. 3 illustrates a simplified schematic of a typical CT-guided needle biopsy (not drawn to scale). In FIG. 3, the left and right panels demonstrate axial and coronal projections, respectively. In this example, a target must be approached at an angle to avoid other important anatomic structures that are labeled “Avoid”. An angle between the needle trajectory and a vertical line from the target lesion (plumb or perpendicular to the floor) is called the “target angle”. In this example, the target angle is 45 degrees. An angle between an opposite needle end (with a hub) and a line parallel to the floor is called the “horizon angle” and should be equal to 180 degrees minus the target angle. In this example, the horizon angle is 135 degrees. In FIG. 3, s is the surface distance from vertical plumb line and d is the depth of penetration to the target.

The current practice is to maintain the target angle in the axial plane while angling in the z-axis is generally avoided as the needle is advanced deeper towards the target. Direct vertical or horizontal orientations for needle placement without oblique angulation are simpler, but have relationships to the floor that the operator also must maintain as the needle is advanced (i.e., horizon angles of 90 and 0 degrees, respectively). Thus, the primary challenge is to prevent or minimize discordance between the planned needle trajectory (see FIG. 3) and an actual needle course throughout the duration of the needle and device placement, and the overall image-guided procedure.

Needle deviations or needle angle errors that occur at or near the skin surface often only become apparent once the needle has traversed deep into the patient. Without any visual reference, the operator may unconsciously alter a correct needle angle or deviate further from the correct needle angle as it is advanced deeply or as the needle encounters tissue interfaces. Correction of a needle angle at depth is only possible for small needle angle errors, as corrections often require withdrawal, adjusted needle angle and reinsertion. This process may require several iterations that further increase tissue injury and, in the context of x-ray or CT guidance, increase patient exposure to ionizing radiation. In the context of any imaging guided procedure (including MM) iterations increase the costs, procedure time and duration of exposure to procedure-related risks for the patient. Thus, it is critical to get the needle angle correct while the needle is at the surface or only superficially placed within the tissue.

Needle or medical device deviations remain common for several reasons. First, the operator must translate angle and depth measurements on the 2-dimensional axial CT images onto an accurate needle target angle on the surface of a patient (with some respiratory motion even when the patient is cooperative). Second, because it is not always possible to view the needle directly orthogonal to the floor or axis of the CT scanner during the procedure, parallax error also can affect true needle position. Third, as the needle is advanced, changes in tissue density (e.g. between fat and muscle) can deflect the needle. The likelihood of encountering error in the actual needle course increases when the target structure is small and/or deeper from the surface, yet these circumstances are often the reason for using image guidance in cases such as a 10-mm retroperitoneal lymph node adjacent to the abdominal aorta 12 cm deep to the surface similar to FIG. 1B.

To minimize error, needle trajectories are planned to be true vertical or horizontal if at all possible. See, e.g., FIG. 1A. When angulated trajectories are necessary to avoid other anatomic structures, the angle is prescribed in only one plane (usually the axial plane as illustrated in FIG. 3) to minimize the potential for compounding error. There are two common ways a needle deviates from the planned trajectory that occur either in isolation or together, hereafter referred to as X-tilt and Z-tilt. As seen in FIG. 4, X-tilt (1^{st} column) occurs when the needle enters the patient too steep (1) or shallow (2) in the axial plane with respect to the planned ideal trajectory (dashed line) to reach the target. Incorrect needle position for X-tilt is recognized in the axial projection, but the other projections usually look normal on images the operator can obtain. For example, subtle needle shortening or lengthening can be difficult to recognize in the coronal projection. Z-tilt (2^{nd} column) occurs when the needle enters the patient with an abnormal angle towards the feet (caudal (3)) or head (cranial (4)) with respect to the planned trajectory. Z-tilt is most evident in the coronal and sagittal projections, but can often be seen in the axial plane when the entire needle is not visualized (e.g. missing superficial needle component in FIG. 1E). These errors are only recognized with imaging in certain planes after they occur.

The magnitude and frequency of needle deviations are subject to an operator's spatial reasoning ability, experience and hand-eye coordination, yet even with highly experienced users, needle position often must be adjusted during the procedure. With the current state of the art, this is an expected component of the procedure at least somewhat mitigated by using image guidance, however the iterative adjustment of needle position and advancement has some disadvantages. Needle placement error can injure anatomic structures leading to undesirable hemorrhage and/or vascular, solid organ or bowel injury. More commonly, needle repositioning increases the volume of tissue traversed by the procedure needle leading to more tissue injury and/or patient pain. Adjustments increase procedure time, which affects patient comfort and the duration of the patient's exposure to conscious sedation, as well as decreased throughput to the detriment of patient wait times and practice revenue. Adjustments also require more imaging, which in the context of x-ray or CT guidance, increases a patient's exposure to ionizing radiation. Finally, the procedure can fail to sample the desired target for treatment or diagnosis.

Many technical solutions have been proposed to improve the safety and efficiency of image guidance during medical procedures over the past 25 years. These include various handheld, stereotactic or robotic devices; augmented visual overlay; and laser, electromagnetic or camera tracking guidance. Although these solutions propose innovative methods for improving the safety and efficacy of image-guided interventions, many of these solutions are expensive or not widely available, and have so far proven difficult to realize widely in clinical practice.

The current state of the art is to direct needle placement using an iterative cycle of needle movement and image guidance, but there is a delay in feedback to the operator from imaging after the needle is manipulated. This delay in feedback significantly lengthens the duration of procedures. In all guidance devices previously proposed, the device holds the needle and prescribes the angle in that the operator inserts the needle through the device, instead of relying on the operator to keep the angle steady by hand.

Robotic systems have been proposed to be placed next to the patient in the imaging suite, but these are designed more to replace or supplement for an experienced operator rather than enhance their abilities. A separate robotic system may prove cumbersome, complicated, expensive and unable to adjust for patient movement during the procedure without repeat setup imaging. Similarly, “brain lab” navigation systems are in common use, for example, at New York University for neurosurgery. However these brain lab navigation systems require extensive preoperative imaging, significant computation and modeling prior to procedures with stereotactic equipment. This is inconsistent with the typical patient presentation and workflow for procedures outside brain tumor resection. These systems are expensive to implement and require additional imaging on a separate occasion. Further, unlike the brain, other regions of the body have more periodic movement over the time that would degrade the fidelity of pre-operative imaging for these systems. Many image-guided procedures also are done on patients who may not be amenable to the highly controlled settings required for the pre-procedure imaging.

Laser fiducials on the needle have been proposed, but these may require a target for the laser projection that may need to be away from the patient or become cumbersome overlying the site of the procedure. The operator or other objects, including the patient, could obstruct the laser reaching the fiducial target.

Real-time ultrasound guidance may work, but only on superficial soft tissue anatomic targets in non-obese subjects. Ultrasound-guidance is extremely limited in regions that contain or are adjacent to air or bone.

The prior art includes the following examples of visual feedback mechanisms:

Jost et al., “iPod Touch-assisted instrumentation of the spine: A technical report,” Neurosurgery, Vol. 73, Operative Neurosurgery 2, pages ons233-ons237 (2013) reports using an iPod Touch in a sterile cover and lining it up with a pedicle screw drill shaft to achieve the correct angle for screw placement. The angles are reported using a freely-available Apple application. This is a bulky impractical method for most imaging-guided procedures since it requires users to hold the Touch against the drill while performing the procedure. The device in this circumstance also requires an additional covering for sterility. There is no dynamic user defined angle or margins for error or auditory feedback. A user must look at the screen oriented orthogonal to the long axis of the needle, drill bit, pedicle screw or other medical device during placement—this increases the likelihood of error or over-corrections, as the line of the site with the medical device is not maintained. Alternatively, the requirement for visual feedback in a different plane or display orientation necessitates at least two operators. In addition, Jost's approach requires a large drill surface to align the Touch and obtain measurements.

Howard et al., “An electronic device for needle placement during sonographically guided percutaneous intervention,” Radiology, Vol. 218, Issue 3, pages 905-911 (2001) reports use of a two-part image guidance system for use in ultrasound guided procedures. The components involved are an attachment to each of a needle probe and to the needle/device itself, in addition to a software package that must be uploaded to the ultrasound machine. Although easily attachable to both the needle and the ultrasound probe, the biggest disadvantage for adaption of this device is the fact that it can only be used in conjunction with ultrasound. No other modality is available for use. Furthermore, it must be used with image guidance rather than any device/needle-based intervention.

Tiesenhausen et al., “A new mobile and light-weight navigation system for interventional radiology,” International Congress and Exhibition “Computer Assisted Radiology and Surgery” (CARS), International Congress Series 1281, pages 412-417 (2005) describes use of a navigation camera and trackers, in conjunction with a laptop that serves as both the display and navigation system/software for needle placement. This approach requires point-point calibration for the workflow, as well as integration with a software package making ease of implementation somewhat laborious. This system would require direct visual line of sight between the navigation camera and the trackers. In addition, the instruments themselves are not able to be retrofit on existing devices and needles. Furthermore, the display is not user friendly or intuitive, as described in the study. Finally, there is no significant data that proves that this instrument in fact decreases time or radiation exposure.

Kim et al., “CT-guided liver biopsy with electromagnetic tracking: results from a single-center prospective randomized controlled trial,” American Journal of Roentgenology, Vol. 203, No. 6, pages W715-W723 (2014) describes use of electromagnetic (“EM”) tracking during CT-guided biopsy, as compared to biopsy without such electromagnetic tracking in 50 patients with liver lesions. The study demonstrates that electromagnetic tracking has a significant impact on metrics for CT-guided procedures such as number of scans, effective radiation dose, number of manipulations per procedure, and scan time. This particular system, while highly effective, requires multiple cumbersome additional pieces of equipment to be set up within the CT-scan room, particularly due to the EM field generator and shielding requirements. Such a setup also would be impossible with MRI systems and may interfere with other medical equipment, such as anesthesia equipment for procedures requiring sedation or general anesthesia. In addition, there is an additional software package that must be applied to the PACS in order to make use of this system.

A need exists for improved technology that is more practical and allows for improvement of the precision and speed of image-guided needle placement to minimize the risks of needle deviations from the planned trajectory.

### SUMMARY

One embodiment of the invention relates to an electronic position guidance device that is configured to attach to a medical device having a first end configured for percutaneous insertion and a second end configured to remain exterior to a patient's skin. The device includes an orientation attachment configured to detect an orientation and a position of the medical device, a processor configured to receive orientation and position information from the orientation attachment and determine a degree to which an actual insertion angle of the medical device deviates from a target angle and/or a degree to which an actual insertion depth of the medical device deviates from a target insertion depth, a display configured to visually convey to a user, in real-time via graphic, text and/or color, the orientation and the position of the medical device, and a speaker configured to audibly convey to the user, in real-time via sound, the orientation and the position of the medical device.

Another embodiment of the invention relates to an electronic position guidance device that is configured to attach to a medical device that does not enter the patient's body percutaneously or otherwise. Instead, the medical device may be configured to act upon a second object disposed within or contacting the patient's body. The device includes an orientation attachment configured to detect an orientation and a position of the medical device, a processor configured to receive orientation and position information from the orientation attachment and determine a degree to which an actual insertion angle of the medical device deviates from a target angle and/or a degree to which an actual insertion depth of the medical device deviates from a target insertion depth, a display configured to visually convey to a user, in real-time via graphic, text and/or color, the orientation and the position of the medical device, and a speaker configured to audibly convey to the user, in real-time via sound, the orientation and the position of the medical device.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The disclosure will become more fully understood from the following detailed description, taken in conjunction with the accompanying figures, in which:

FIGS. 1A-1F illustrate various embodiments in which image guidance can be used for needle-based procedures. FIG. 1A shows a CT-guided pulmonary nodule biopsy and/or ablation. FIG. 1B shows a retroperitoneal lymph node biopsy. FIG. 1C shows a bone biopsy.

FIG. 1D shows an intra-abdominal or intra-pelvic abscess drainage. FIG. 1E shows an ablation procedure of tumors of visceral abdominal organs (liver or kidney tumors). FIG. 1F shows a lumbar puncture for cerebrospinal fluid studies, intrathecal chemotherapy or CT myelogram.

FIG. 2 illustrates a typical process for a CT-guided procedure for a left L4 transforaminal epidural block for lower back pain.

FIG. 3 illustrates a schematic of a typical CT-guided needle biopsy.

FIG. 4 illustrates X-tilt and Z-tilt errors in needle angle at a surface entry point in an axial, a coronal and a sagittal projection.

FIG. 5 illustrates a schematic diagram of an electronic position guidance device with real-time auditory and visual feedback.

FIG. 6 illustrates an electronic chip of the electronic position guidance device of FIG. 5. The electronic chip has an accelerometer that may be fitted to a needle or other medical device to assist in position guidance.

FIG. 7 illustrates an example of an interface for a user to predefine a relative position of a needle and microchip plane relative to pure horizontal or vertical.

FIGS. 8A-8C illustrate examples of potential visual display feedback during image-guided needle placement using the position guidance device of FIG. 5. FIG. 8A illustrates an example in which the needle is in the correct position, and the dot overlies the user defined cross-hairs (which also illustrate deviation from pure vertical). FIG. 8B illustrates an example in which needle Roll is erroneous and the dot's position relative to the cross-hairs conveys the direction and magnitude of error. FIG. 8C illustrates an example in which there is an error in the needle Pitch, and the dot can be seen to deviate from the desired position relative to the cross-hairs.

FIG. 9 illustrates an example of the electronic chip of FIG. 6 fitted to a needle.

FIG. 10 illustrates another example of the electronic chip of FIG. 6 fitted to a needle.

FIG. 11 illustrates an example of the electronic position guidance device of FIG. 5, including the electronic chip of FIG. 6 fitted to a needle and a display screen configured to provide potential visual display feedback.

FIG. 12 illustrates another example of the electronic position guidance device of FIG. 5, including the electronic chip of FIG. 6 fitted to a needle and a display screen configured to provide potential visual display feedback.

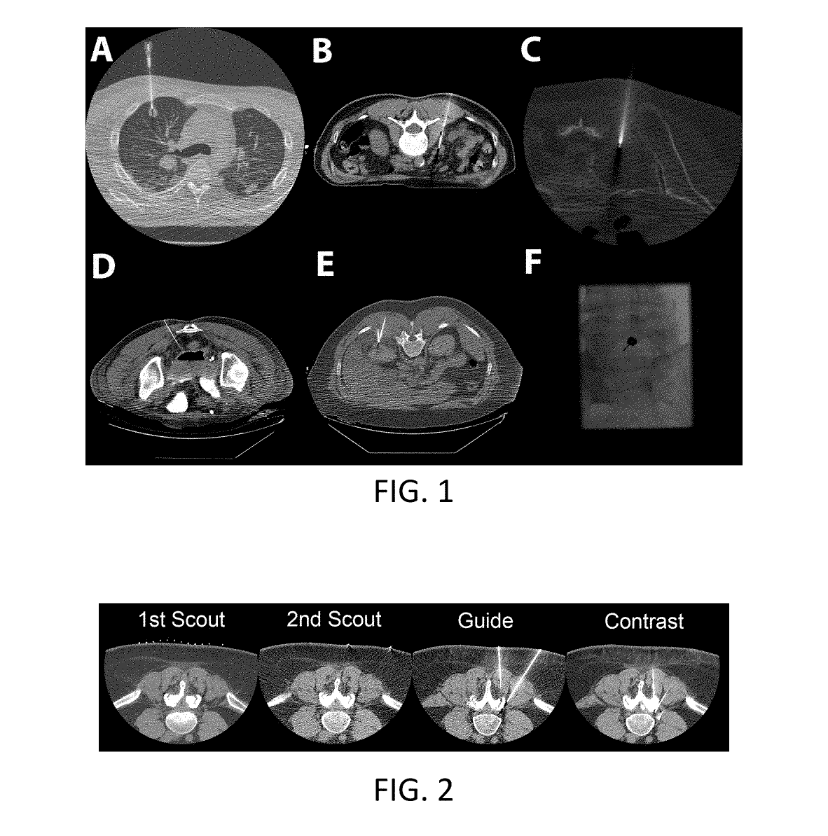

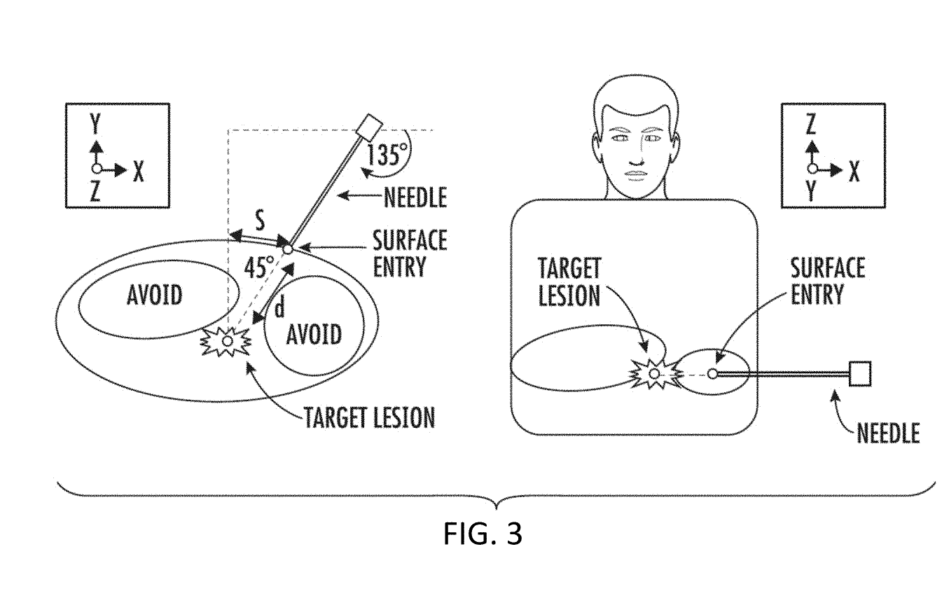

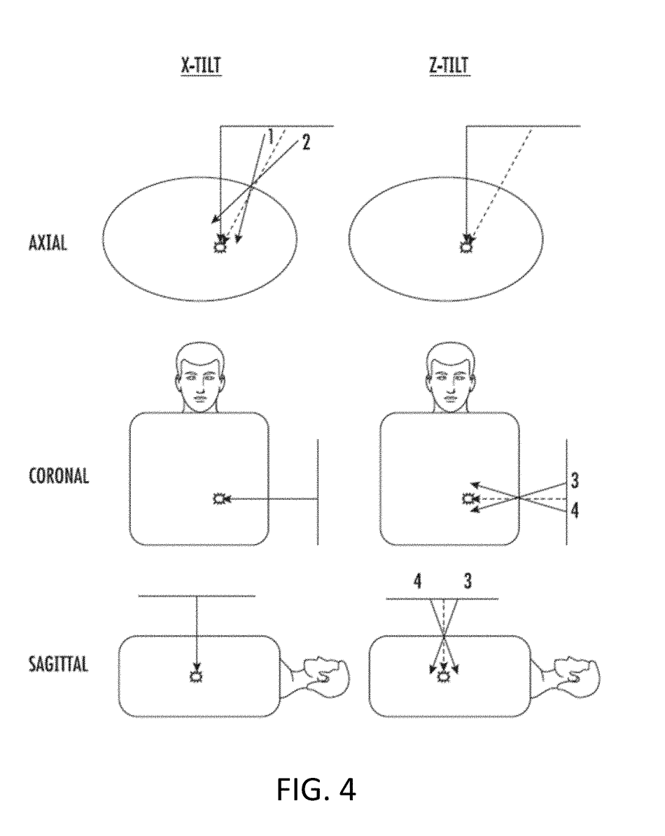

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

For the purposes of this description “x-tilt” and “z-tilt” define errors in needle or device position, whereas “pitch” and “roll” are the desired angles of the device relative to pure vertical and horizontal for correct placement. Hence x-tilt is the amount of error in pitch, while z-tilt is the amount of error in roll.

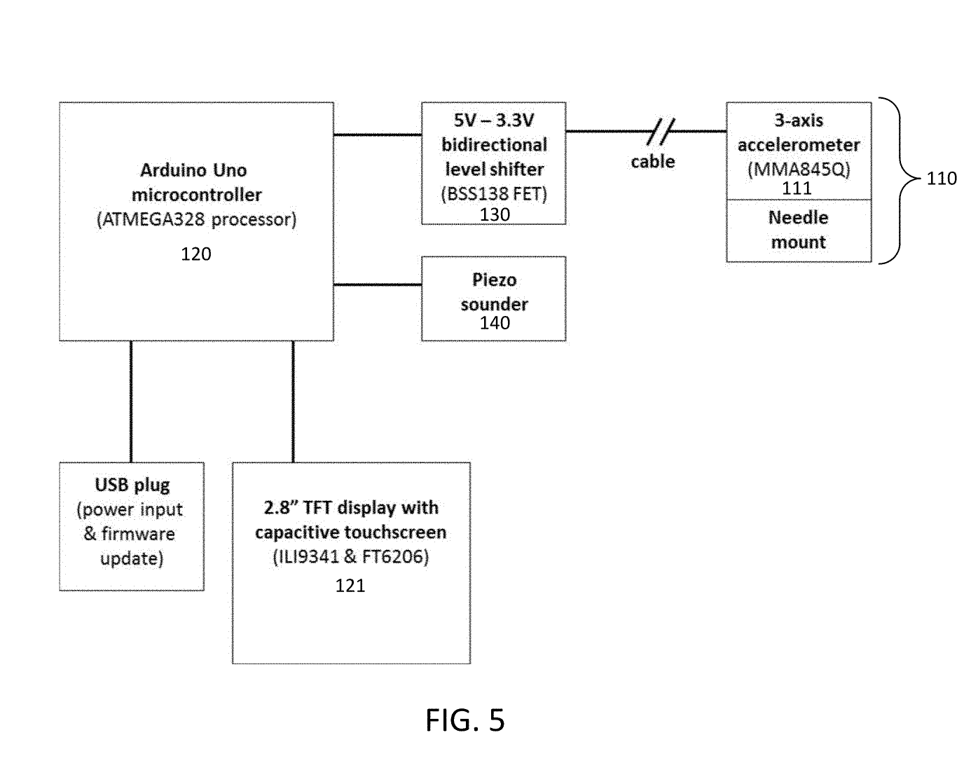

Referring to FIGS. 5-8C, an electronic position guidance device 100 may be attached to a medical device 200 having a first end 210 configured for percutaneous insertion and a second end 220 configured to remain exterior to a patient's skin. The medical device 200 may be, for example, a needle. The electronic position guidance device 100 represents a novel modification to medical devices and needles 200 that allows the user to actively adjust the orientation of the needle 200 relative to the patient, with the assistance of dynamic, real time auditory and visual feedback mechanisms. The electronic position guidance device 100 may be easily applied (i.e., retrofit) to existing devices and needles 200. The electronic position guidance device 100 provides real-time feedback to the user regarding the angle of device/needle 200 relative to the vertical, as well as the degree of error relative to a target orientation. The electronic position guidance device 100 also provides feedback regarding the angle of the device/needle 200 relative to the orthogonal plane, which is usually the “horizontal” level, perpendicular to the ground. As the needle 200 is advanced forwards or backwards, the electronic position guidance device 100 relays auditory and visual feedback in accordance with the degree of deviation from the pre-programmed angle relative to the vertical and horizontal, with changes in sound proportional to the degree of error from the pre-determined angles in each respective dimension.

In one embodiment, the electronic position guidance device 100 includes three components configured to communicate with one another via small wire attachments, infrared or radiowaves (e.g., Blue-Tooth®/WiFi™). All three components are configured to be manufactured to support repeated use after treatment. Further, if needed, all or some components of the electronic position guidance device 100 may be sterilized repeatedly for repeat use, for example, with ethylene oxide gas or autoclave sterilization.

Component 1—Orientation Attachment

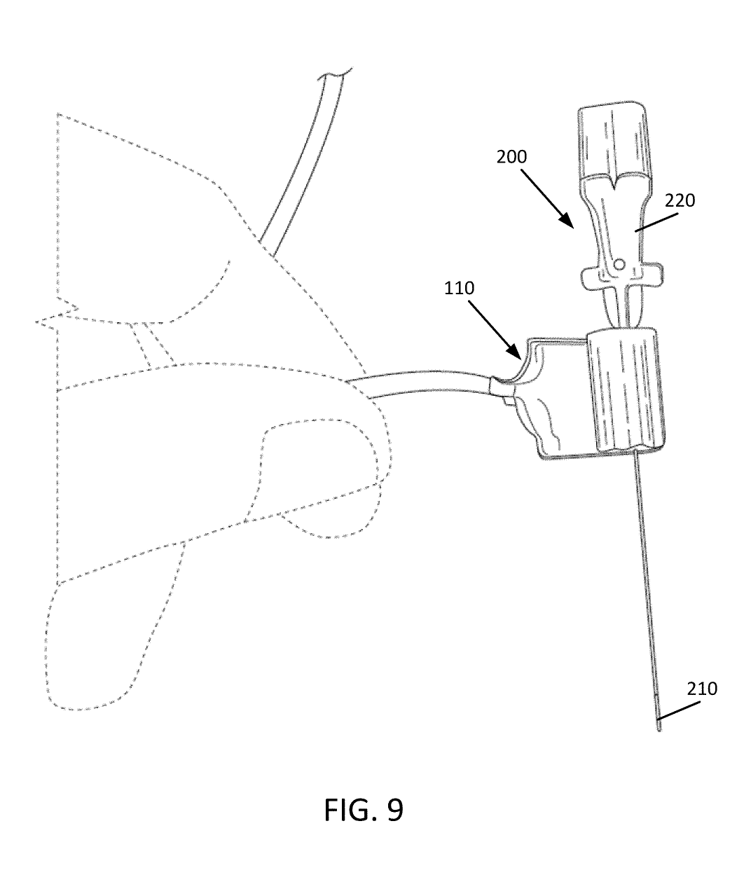

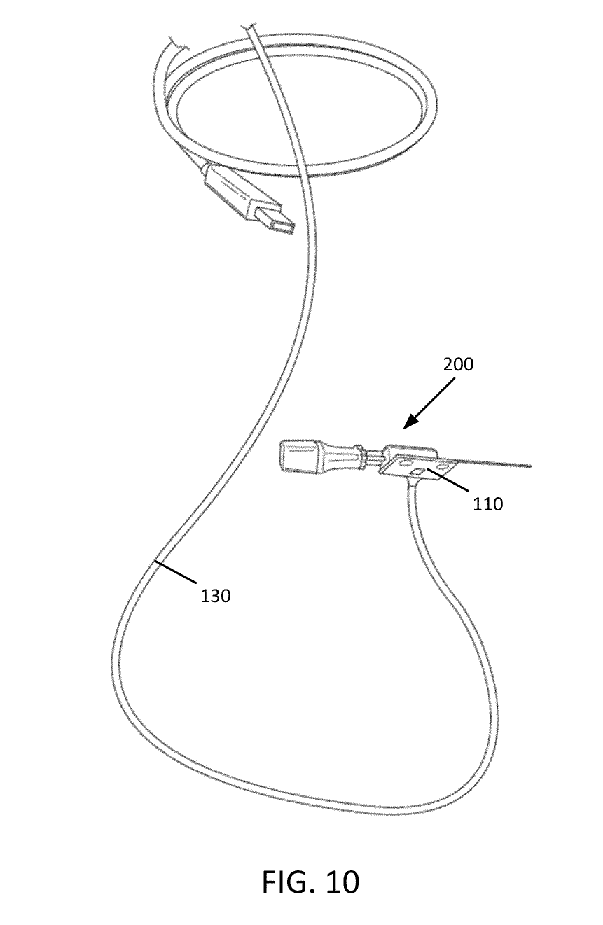

A small, lightweight electronic orientation attachment (“orientation attachment”) 110 for the needle or medical device 200 contains an accelerometer 111 and a small indicator light (see FIGS. 9 and 10). The orientation attachment 110 is configured to provide data on the needle/medical device 200 orientation to a computation and data input/output module.

Component 2—Master Device

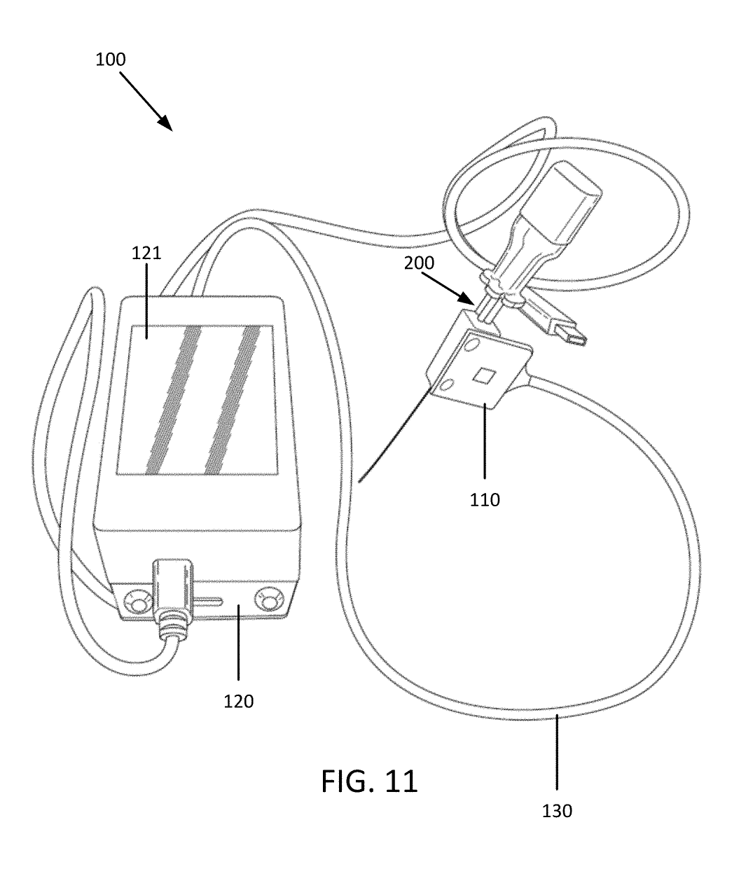

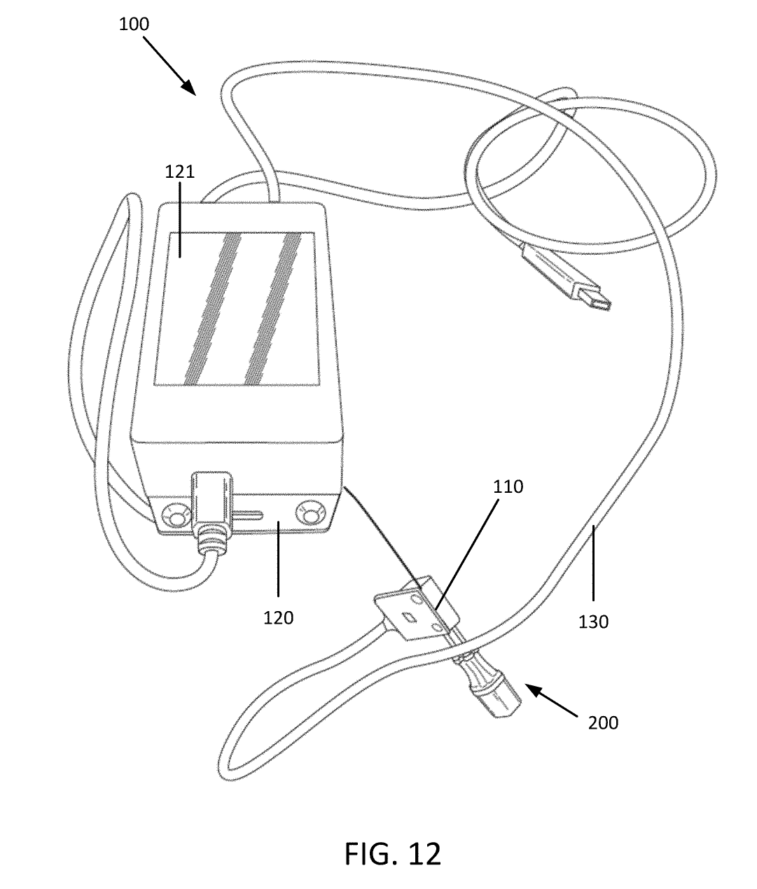

A separate master sensor device (“master device”) 120 for user-input is configured to provide visual and auditory feedback via a display screen 121 (see FIGS. 11 and 12) to the operator regarding real time needle 200 orientation and position. The master device 120 is programmed to perform all data analysis and computation based on received data from the orientation attachment 110. Using the master device 120, a user can use a touchscreen 121 input mechanism to input a targeted pitch angle (“target angle”) as well as the tolerated error. This master device 120 can be configured for user input in a variety of ways, for example, the master device 120 may include a remote control, touchscreen, dial or other forms of input. The master device 120 also could supply display information to other systems, such as large monitors in the procedure room or glasses the operator can wear during the procedure.

Component 3—Connection Wire

A connection wire 130 is configured to connect the orientation attachment 110 and the master device 120.

In one embodiment (see FIG. 5), the electronic position guidance device 100 uses a master device 120 to read data from an orientation attachment 110 (i.e., an accelerometer 111) mounted on a needle 200. For example, the master device 120 may be an Arduino Uno (ATMEGA832-based microcontroller) configured to read data from an orientation attachment 110 such as a 3-axis accelerometer 111 mounted on the needle 200. The accelerometer 111 may be connected to the Arduino Uno 120 via a connection wire 130 such as a 5 v-3.3 v signal level shifter and a long (1-2 m) cable.

A visual display with a touchscreen 121 may be connected to the master device 120. For example, a 2.8″ TFT visual display with touchscreen 121 may be connected to the Arduino Uno 120, for visual output (measured angle) and user input (selecting target angle, tolerance and audio mute). Audio output may be generated, for example, by a piezoelectric sounder 140 connected to the Arduino Uno 120. The electronic position guidance device 100 may receive power via the Arduino Uno USB port. The USB port may also be used for updating the electronic position guidance device's firmware.

The orientation attachment 110 will communicate needle 200 orientation and position information in 3-D space relative to datum positions (e.g., a horizontal and a target orientation, registered at the beginning of the procedure), to the master device 120. This may be implemented, for example, using a light-weight microchip position sensor 111 (e.g., a sensor containing a 3-axis accelerometer, a gyroscope and magnetometer sensors) physically attached to the biopsy needle or medical device 200. In some embodiments, attachment of the electronic position guidance device 100 to the needle or medical device 200 is permanent. In other embodiments, the electronic position guidance device 100 may be removably attached to the needle or medical device 200 via standard Luer-lock or slip-tip needle configurations, to the bore of the needle 200 itself, or by any other suitable connection.

After an initial auto-calibrate step at the start of the procedure, changes in needle 200 orientation and position are recorded based on information reported back from the orientation attachment 110 to the master device 120. The master device 120 may convey the needle 200 orientation and position information to the user via a graphical and/or textual display. For example, the graphical and/or textual display may read “−3°,” indicating that the needle 200 is 3 degrees too steep.

In addition to the graphical and/or textual display 121, or as an alternative to the graphical and/or textual display 121, the master device 120 may convey needle 200 orientation and position information to the user using a color display. For example, the colors red, yellow, and green may be used to help indicate the margin of error in needle 200 position or orientation in user defined margins (e.g. +/−5 degrees vs +/−10 degrees for angle orientation). This information is relayed in terms of “pitch” and “roll” which indicate orientation angles relative to the vertical and horizontal, respectively, similar to “x-tilt” and “z-tilt.” In some embodiments, an acceptable margin of error may be conveyed in green text or with a green light, an unacceptable margin of error may be conveyed in red text or with a red light, while a margin of error that is unacceptable, but close to the acceptable margin of error may be conveyed in yellow text or with a yellow light. In other embodiments, the color display may only include two colors (e.g., red and green) to indicate unacceptable and acceptable margins of error. In some embodiments, the colors may represent angles that are too steep or shallow in different orientation planes, or reflect degrees of steep error. In other embodiments, color changes could also be associated with deviation of the needle angle during active movement of the needle 200. These embodiments are not necessarily mutually exclusive. The device may be configured to offer a variety of different user-defined color schemes to convey device position and angle information.

The master device 120 may also generate auditory signals (frequency, and tone) based on user-defined tolerances and types of feedback. The auditory signals may also differentiate between errors of “pitch” versus “roll” in situations where the rotation orientation of the needle 200 is significant. A signal emitter/sensor on the master device 120 may send information back to the orientation attachment 110 to drive visual feedback directly from the small attached indicator light (i.e., color and/or flashing frequency, which may also be pre-defined by a user or manufacturer to report error tolerances).

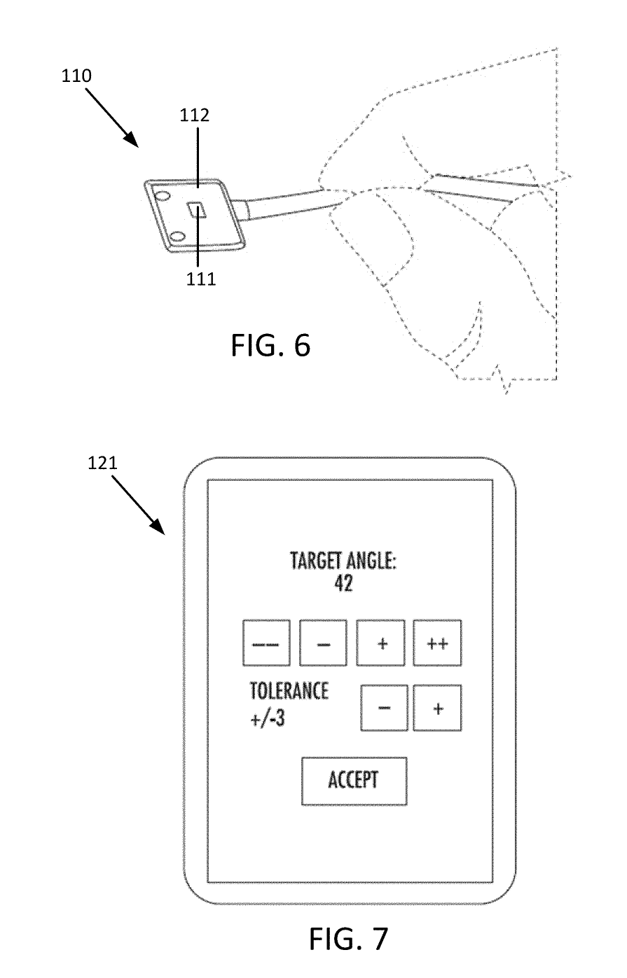

As seen in FIGS. 6 and 9-12, a small electronic chip 112 with an accelerometer 111 (<1 cm² surface area and <5 g) may be fitted to a needle or other medical device 200. Communication to a second feedback device may be via wire wireless (e.g., infrared, bluetooth, or wifi). The electronics provides information about the movement of a chip plane relative to horizontal in two dimensions. If the chip 112 is attached to the needle or medical device 200 at a predetermined angle, a user defined relative angle of this plane to the horizontal may be used to control the angle of the needle 200 relative to an anatomic target in the body. Deviations from this angle may then be reported back to the user as the needle or other device 200 is moved through space. The chip 112 may also be configured to detect movement through space in a third dimension (for example, by inclusion of gyroscope and magnetometer sensors in addition to accelerometers, with simple geometric corrections for angulation of needle 200 relative to true horizontal or vertical), hence detecting the depth of penetration into an anatomic structure.

FIG. 7 is an example of an interface for the user to predefine the relative position of the needle 200 and microchip plane relative to pure horizontal or vertical. Based on the relative angle of the desired needle path to the anatomic target observed on a medical image (ultrasound, MRI, CT or other), the user may prescribe a desired angle for needle entry in 2 axes (“Pitch” and “Roll”). The tolerance for errors and visual/auditory feedback may also be user defined—hence in a shallow target without adjacent critical structures, a tolerance of 3-5 degrees may be acceptable, whereas near a vital structure, such as the abdominal aorta, a tolerance limited to only 1 degree may be desired.

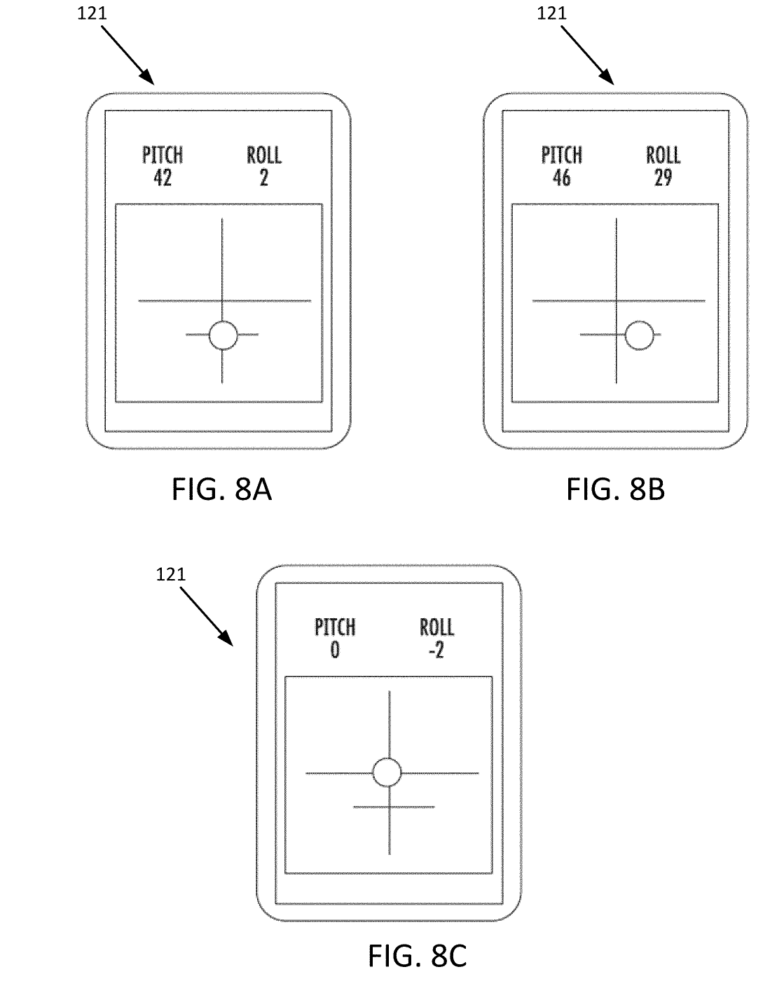

FIG. 7 is an example of a potential visual display feedback during image-guided needle placement using the electronic position guidance device 100. In the examples of FIGS. 8A-8C, the target angle of entry for the needle 200 is 42 degrees Pitch and 0 degrees Roll (where Pitch and Roll are orthogonal angles to one another similar to “x-tilt” and “z-tilt”). When the needle 200 is in the correct position, the dot overlies the user defined cross-hairs (which also illustrate deviation from pure vertical) and the dot is colored green (see FIG. 8A). In this example, when needle Roll is erroneous the dot turns yellow and its position relative to the crosshairs conveys the direction and magnitude of error (see FIG. 8B). Conversely if there is an error in the needle Pitch, the dot is red and can be seen to deviate from the desired position relative to the crosshairs (see FIG. 8C). Different audio tones also can be associated with correct position, and small or large needle angle deviations. Tones also could be unique for error in Pitch, Roll or a combination of both angles. Tones and dot colors could be varied according to user preferences and defined tolerances. The examples of potential visual display feedback illustrated in FIGS. 7 and 8A-8C may be displayed, for example, on the display screen illustrated in FIGS. 11 and 12.

The electronic position guidance device 100 differs from a simple smartphone-based attachment (e.g., as described in Jost above) for the following reasons. A cell phone is bulky and heavy, whereas this device spatially separates the sensor from the rest of the device. The advantage of this is that the sensor can be firmly attached to the needle 200, but does not interfere with needle insertion because of its weight or size. Having a sensor 111 physically attached to the needle 200 ensures that the user has temporally accurate and continual feedback throughout the needle insertion procedure. Thus, the operator can focus solely on the direction and movement of the device without also needing to focus on maintaining the correct the orientation and attachment of the smartphone-based attachment to the device. Further, the combination of the audio feedback and the lightweight sensor means that the user can concentrate on looking at the needle or device 200 and the position relative to the patient or target structure, rather than having to look at a phone. Thus, continual, accurate orientation information is received and utilized while the user looks at the needle 200 and patient. This allows the combination of both visual inspection of the needle entry and visual feedback from the sensor at the same time.

In other embodiments of the electronic position guidance device 100, two modifications may be made that allow utilization of the sensor/module as well as the master device 120.

In the first modification, the components may be separated such that the orientation attachment 110 remains attached to a wire. However the master device 120 may be embodied by a smartphone/tablet. In this embodiment, the orientation attachment 110 plugs directly into a smartphone or tablet. However, the software that is utilized for the master device 120 would be transformed into an application that can be pre-installed on a smartphone or table device. The user would input all target angle and tolerance information into the application (pre-installed onto the smartphone/tablet), while use of the orientation attachment 110 remains the same as described above. This embodiment may not be the preferred method of use, given HIPAA concerns around using a personally hand-held smartphone or iPad/tablet and sterility issues, however, the key information for the procedure as concerns this invention (i.e., depth of target, desired pitch and roll, error tolerances and feedback choices) does not require any patient identifying information. Data from the procedure also could be archived during or after completion and added to the patient medical record. If smartphones or iPads/tablets are for general purpose as designated by the institution and user, this embodiment of the device would work well.

In the second modification, the orientation attachment 110 may work via a Bluetooth or WiFi/internet/intranet signal thereby eliminating the necessity for the wire connecting this component to the master device 120 (or smartphone/iPad/tablet if the above modification is utilized). Simple modifications to the software installed on the master device 120 or downloaded onto the smartphone/iPad/tablet can be made to allow for this change.

Immediate and future applications of the electronic position guidance device 100 described in the embodiments above include use during any medical procedure during which a needle (or other interventional/surgical medical device) 200 is used. These include image-guided injections, biopsies and ablation procedures and fluid collection/abscess drainages. Additional examples include vascular access procedures as well as access procedures into organs such as the kidney and biliary system for percutaneous nephrostomy tube or cholecystostomy/biliary tube placement. As another example, a modification of the device also could be used for spinal hardware pedicle screw insertion. The electronic position guidance device 100 potentially would only require imaging to plan the trajectory and then final images to confirm position once the needle 200 was predicted to reach the target. This would make procedures dramatically faster, safer, less imaging intensive (e.g. less radiation from CT) and increase operator confidence. The electronic position guidance device 100 also may prove beneficial to non-image guided procedures such as intra-cranial drain placement and laparoscopic trochar placement.

Another modification of the invention includes providing a separate setting on the master device 120 that can be toggled such that the operator knows if the needle 200 has moved significantly in any dimension. For certain procedures, while achieving a needle position, angle and/or depth is typically the initial objective, maintaining that precise position without advancing or retracting the needle 200 during the second part of the image-guided procedure is equally as important. These exchanges or manipulation at the hub of the needle 200 require absolute stability of the needle position, but an operator will only know if that has been achieved by repeat imaging or if a patient reports symptoms. In an anesthetized, sedated patient, or in a patient where the local tissues have been anesthetized, feedback can only be obtained by repeat imaging. This is not always an option since the exchange or injection requires continuous activity or a series of rapid, sequential steps. Imaging in this instance would be disruptive. Further, in an operating room, repeat imaging is rarely possible and far more burdensome to obtain. For example, intraoperative MM for neurosurgery cases requires thirty minutes to perform.

To illustrate the importance of maintaining a precise needle position without advancing or retracting the needle 200, the following example is provided. During spinal cervical nerve root block, the needle 200 is carefully advanced such that the tip is placed directly adjacent to the a C5 nerve root for injection of a lidocaine/steroid mixture—typically for relief of neck pain due to foraminal stenosis. Because of the design of most commercially available needles 200, once the needle position is correct, an inner stylet is removed so that solution can be connected to the outer needle sheath via a luer lock screw top. Additionally, often times a contrast injection is required (also requiring removal of the inner stylet of the needle 200 and connection to a contrast filled syringe) prior to injection of steroid/lidocaine mixture in order to confirm the precise location of the needle tip 210. While precision and accuracy are paramount during any intervention, it should be obvious in cervical nerve root interventions, where 2-3 mm inadvertent advancements of the needle 200 beyond the intended position might result in severe neurologic complication, such as spinal cord puncture (with resultant paralysis) or vertebral artery puncture and associated vessel dissection with ischemic stroke. In this modification, the master device 120 may emit a signal after the toggle switch has been set to “on” so that any advancement or retraction of the needle 200 results in a visual and/or an auditory signal to the operator. It should be noted that similar to the description of “tolerated angle error” above, a certain degree of “leeway” can be set by the operator—for example 1-2 mm beyond or before the needle position. Thus, the current invention may be used to help prevent movement of the device or to provide immediate feedback indicating that the needle or device 200 is being inadvertently moved during this portion of the particular procedure.

Another embodiment of the invention includes providing an additional component comprising an additional sensor module (e.g., 3-axis accelerometers, magnetometers or gyroscopes) that may be attached to the patient near the site of the procedure and/or at other body points to provide feedback for a) the relationship of the device to other key anatomic structures, and b) detected patient movement that requires altering or re-assessing the desired angle or depth of penetration of the needle/other medical device 200. In the former example, correlation of orientation and position information from both patient and needle 200 may afford a higher degree of accuracy in attaining and maintaining position of a needle 200 relative to a key anatomic feature within the body. In the latter example, the device may serve to detect undesired patient movement macroscopically or within the body region undergoing the procedure, and also provide accurate data for real-time adjustments to the target angle and/or depth of penetration by the user and/or master device 120. These adjustments could be automatically done by the master device 120, or require the user to redefine the goal orientations.

The electronic position guidance device 100 described in the embodiments above improves accuracy and safety of needle or medical device 200 placement under image guidance by way of (a) providing both audio and visual sensory components for real time feedback of needle orientation, (b) providing visual output of needle or medical device 200 position data as a numeric and graphical display, (c) providing visual output of position data via a needle-mounted output (e.g. LED), (d) utilizing lightweight attachments that are not cumbersome to the patient or operator, and (e) having the capability to be utilized in conjunction with standard clinically used needles and medical devices 200. Currently, non-real-time iterative adjustments are made that allow for multiple novel and repeated errors while performing the procedure and therefore the electronic position guidance device described in the embodiments above would reduce the potential for these novel and repeated errors.

The electronic position guidance device 100 involves real-time, dynamic auditory and/or visual feedback, allowing the user to utilize additional intrinsic sensory information to adjust/improve accuracy of angle insertion and advancement. Although several visual real-time feedback mechanisms already exist, the addition of dynamic, auditory real-time feedback, as the user inserts and advances the needle 200, yields an added level of specificity and accuracy to the procedure. Furthermore, as the degree of angle error increases relative to the pre-determined angle, so too does the degree of auditory feedback, thereby providing valuable quantitative, real-time, dynamic data to the operator. As the auditory feedback relies on an electronic sensor to generate data on needle position, position information is simultaneously relayed to the operator via a visual display showing deviation from the target angle in each dimension that is directly connected to the device. Finally, the electronic position guidance device 100 may be utilized in addition to existing or future needle-based modification devices.

Further embodiments of the electronic position guidance device 100 may include means for providing haptic feedback to the operator. For example, the electronic position guidance device 100 may include a small pad configured to be attached to the operator (for example, the pad may be taped or otherwise attached to the operator's wrist or finger) and configured to provide vibration alerts to operator when the needle 200 is moving. In such an embodiment, the small pad is provided separate from the rest of the electronic position guidance device 100, such that a vibration of the small pad does not cause vibration of the medical device 200 to which the electronic position guidance device 100 is attached.

The construction and arrangements of the electronic position guidance device, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, data processing algorithms, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

Embodiments of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on one or more computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate components or media (e.g., multiple CDs, disks, or other storage devices). Accordingly, the computer storage medium may be tangible and non-transitory.

The operations described in this specification can be implemented as operations performed by a data processing apparatus or processing circuit on data stored on one or more computer-readable storage devices or received from other sources.

The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors or processing circuits executing one or more computer programs to perform actions by operating on input data and generating output. The processes and logic flows can also be performed by, and apparatus can also be implemented as, special purpose logic circuitry, e.g., an FPGA or an ASIC.

Processors or processing circuits suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display), OLED (organic light emitting diode), TFT (thin-film transistor), plasma, other flexible configuration, or any other monitor for displaying information to the user and a keyboard, a pointing device, e.g., a mouse trackball, etc., or a touch screen, touch pad, etc., by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

What is claimed:

1. An electronic position guidance device configured to attach to a medical device having a first end configured for percutaneous insertion and a second end configured to remain exterior to a patient's skin, the electronic position guidance device comprising:
an orientation attachment configured to be attached to the medical device and to detect an orientation and a position of the medical device;
a processor configured to receive orientation and position information from the orientation attachment and determine a degree to which an actual insertion angle of the medical device deviates from a target angle and/or a degree to which an actual insertion depth of the medical device deviates from a target insertion depth;
a display configured to visually convey to a user, in real-time via graphic, text and/or color, the orientation and the position of the medical device to which the electronic position guidance device is attached; and
a speaker configured to audibly convey to the user, in real-time via sound, the orientation and the position of the medical device to which the electronic position guidance device is attached.

2. The electronic position guidance device of claim 1, wherein the orientation and the position of the medical device visually conveyed by the display includes the actual insertion angle of the medical device and the target angle.

3. The electronic position guidance device of claim 1, wherein the orientation and the position of the medical device visually conveyed by the display includes the actual insertion depth of the medical device and the target insertion depth.

4. The electronic position guidance device of claim 1, wherein the speaker is configured to audibly convey to a user, in real-time, a degree to which an actual insertion angle of the medical device deviates from a target angle.

5. The electronic position guidance device of claim 1, wherein the electronic position guidance device is attached to a needle.

6. The electronic position guidance device of claim 1, wherein the orientation attachment comprises an accelerometer.

7. The electronic position guidance device of claim 1, wherein a frequency and/or tone of the sound emitted by the speaker is configured to increase or decrease based on a degree to which an actual insertion angle of the medical device deviates from a target angle or a degree to which an actual insertion depth of the medical device deviates from a target insertion depth.

8. The electronic position guidance device of claim 1, further comprising at least one additional sensor module configured to be attached to a patient near a site of the procedure, at a body surface landmark, or a combination thereof.

9. The electronic position guidance device of claim 8, wherein the at least one additional sensor module is selected from the group consisting of 3-axis accelerometers, magnetometers and gyroscopes.

10. The electronic position guidance device of claim 9, wherein the at least one additional sensor module is attached to the body surface landmark, and the processor is configured to correlate orientation and position information from both the body surface landmark and the medical device to attain and maintain a position of the medical device relative to the body surface landmark.

11. The electronic position guidance device of claim 9, wherein the at least one additional sensor module is attached to the patient near the site of the procedure and is configured to detect undesired patient movement, and the processor is configured to correlate orientation and position information from both the patient and the medical device to attain and maintain a position of the medical device relative to the patient.

12. The electronic position guidance device of claim 5, wherein the processor includes a toggle switch configured to be set “on” or “off,” and when the toggle switch is set “on,” the processor is configured to cause the speaker to emit an auditory or visual signal when the needle is advanced or retracted.

13. The electronic position guidance device of claim 1, wherein the display and the speaker are not physically attached to the medical device.

14. The electronic position guidance device of claim 1, wherein at least one of the display and the speaker is physically attached to the medical device.

15. The electronic position guidance device of claim 1, wherein both the display and the speaker are physically attached to the medical device.

16. The electronic position guidance device of claim 1, wherein the display comprises a monitor in the procedure room or glasses configured to be worn by an operator during a procedure.

17. The electronic position guidance device of claim 1, wherein after the medical device is oriented and positioned at the target angle and/or at the target insertion depth, the processor is further configured to determine whether the target angle and/or the target insertion depth are maintained during a procedure.

18. The electronic position guidance device of claim 17, wherein the processor is configured to issue a visual and/or auditory alert if the orientation and position of the medical device deviates from the target angle and/or the target insertion depth by a predetermined amount.

19. The electronic position device of claim 18, wherein the predetermined amount is configured to be set by user input received by the processor prior to a procedure in which the medical device is used.

20. An electronic position guidance device configured to attach to a medical device that does not enter the patient's body percutaneously or otherwise, the electronic position guidance device comprising:
an orientation attachment configured to be attached to the medical device and to detect an orientation and a position of the medical device;
a processor configured to receive orientation and position information from the orientation attachment and determine a degree to which an actual insertion angle of the medical device deviates from a target angle and/or a degree to which an actual insertion depth of the medical device deviates from a target insertion depth;
a display configured to visually convey to a user, in real-time via graphic, text and/or color, the orientation and the position of the medical device to which the electronic position guidance device is attached; and
a speaker configured to audibly convey to the user, in real-time via sound, the orientation and the position of the medical device to which the electronic position guidance device is attached,
wherein the medical device is configured to act upon a second object disposed within or contacting the patient's body.
